(19) United States
(12) Patent Application Publication
(10) Pub. No.: US 2026/0363641 A1
(43) Pub. Date: Nov. 19, 2026

(54) METHODS FOR ADAPTING A MAMMOGRAPHY OF A MAMMOGRAPHY SYSTEM

(71) Applicant: Siemens Healthineers AG, Forchheim (DE)

(72) Inventor: Marcus RADICKE, Veitsbronn (DE)

(73) Assignee: Siemens Healthineers AG, Forchheim (DE)

(21) Appl. No.: 19/674,804

(22) Filed: May 12, 2026

(30) Foreign Application Priority Data
May 13, 2025 (DE) 10 2025 118 324.4

(51) Int. Cl. A61B 6/00; A61B 6/04; A61B 6/50; A61B 6/58

(52) U.S. Cl. CPC A61B 6/488; A61B 6/0414; A61B 6/502; A61B 6/544; A61B 6/545; A61B 6/582

(57) ABSTRACT

A method for adapting a mammography of a mammography system is described. In the method, an image region, in which a breast to be mapped is positioned, is sampled using a sensor during the positioning and compression of the breast, is the sensor operating based on a physical active principle without an ionizing effect on patient tissue, wherein preliminary image data is obtained from the image region. The obtained preliminary image data is evaluated with regard to a subsequent X-ray mapping of the breast, wherein adapted values for imaging parameters are obtained for the subsequent X-ray mapping. Finally, the mammography system is adapted based on the obtained adapted values of the imaging parameters. A method for carrying out a mammography is also described. Furthermore, an adaptation facility is described. A mammography system is described, moreover.

### CROSS-REFERENCE TO RELATED APPLICATION(S)

The present application claims priority under 35 U.S.C. § 119 to German Patent Application No. 10 2025 118 324.4, filed May 13, 2025, the entire contents of which are incorporated herein by reference.

### FIELD

One or more example embodiments relates to a method for adapting a mammography of a mammography system. One or more example embodiments refers to a method for carrying out a mammography. In addition, one or more example embodiments relates to an adaptation facility (also referred to as an adaptation system). One or more example embodiments also refers to a mammography system.

### RELATED ART

During a breast examination, in particular in conjunction with early detection of breast cancers, as a rule a mammography is carried out as imaging in which the breast is compressed and is mapped on an X-ray detector with the aid of X-ray radiation. What is known as an “X-ray advance shot” is conventionally used in X-ray-based breast imaging in order to ascertain the optimum exposure parameters for the subsequent actual imaging of the breast. The exposure parameters comprise, in particular, the electric voltage Ua, which is applied in the X-ray tube between anode and cathode, and is measured in kilovolts, and the energy or the heating voltage Uh, with which the cathode is heated and with which the number of emitted photons or X-ray photons or the intensity of the X-ray radiation or the X-ray emission is defined.

Put simply, the most absorbing structure in the breast is sought and the dose of the X-ray emission for the actual imaging of the breast is regulated thereon. Until now, this process has lasted more than one second and is composed, for example, of what are known as two region-growing algorithms. During this time the female patient has to stay in a somewhat uncomfortable situation with their breast already compressed until said calculations are ended and the actual imaging can be started.

### SUMMARY

One or more example embodiments reduces this time in which the female patient is exposed to this compression in order to improve the patient comfort. One or more example embodiments improves the patient comfort during a mammography.

### BRIEF DESCRIPTION OF THE DRAWINGS

The invention is explained below on the basis of exemplary embodiments with reference to the accompanying figures. In the drawings:

FIG. 1 shows a schematic representation of a scenario of a recording of mammography image data from the breast of a female patient,

FIG. 2 shows a flowchart which illustrates a method for adapting a mammography of a mammography system according to a first exemplary embodiment of the invention,

FIG. 3 shows a flowchart which illustrates a method for carrying out a mammography according to an exemplary embodiment of the invention,

FIG. 4 shows a schematic representation of an adaptation facility according to an exemplary embodiment of the invention, and

FIG. 5 shows a schematic representation of a mammography system according to an exemplary embodiment of the invention.

### DETAILED DESCRIPTION

In a method for adapting imaging parameters, in particular exposure parameters, of a mammography system, firstly an image region is sampled in which a breast to be mapped is positioned, during the positioning and compression of the breast, by way of a sensor whose function is based on a physical active principle without an ionizing effect on patient tissue. The sensor therefore functions on the basis of a physical effect, which is implemented without a potentially health-endangering ionizing effect on body tissue. Preliminary image data is obtained from the image region during the sampling process.

Imaging parameters comprise, in particular, the collimation of the X-ray radiation and the activation of the X-ray pixels of the X-ray detector, but also the spectrum of the X-ray radiation and its intensity.

The “sensor” can also comprise a plurality of sensors of the same type or a plurality of sensors partially of different types, with it also being possible for the sensors to be arranged at different positions and/or spatially separated from one another. The sensor can comprise an active sensor and/or a passive sensor. An active sensor comprises an active unit, preferably for the emission of waves, in particular electromagnetic waves, which act on the object to be mapped or sampled. A passive sensor does not have such an active unit.

Preferably, the sensor function of the sensor is based on receiving and evaluating waves, preferably electromagnetic waves, from the object to be sampled, the wavelength of which is significantly above the wavelength of ionizing radiation based on waves, in particular X-ray radiation.

In the electromagnetic spectrum, non-ionizing waves of this kind comprise wavelengths of more than approximately 250 nm.

Conversely, only cosmic radiation, gamma radiation, X-ray radiation and shorter wave ultraviolet radiation have sufficient quantum energy to detach electrons from the atomic shells and thus also separate covalent bonds.

By contrast, radio waves, radar waves, microwaves, infrared radiation or visible light do not comprise any ionizing radiation because they cannot permanently change or even break up molecules (apart from specific, light-sensitive substances). Said types of waves are therefore suitable for the sensor used in the inventive manner. Molecules, which would be broken up by such low-energy photons, could not exist under normal conditions.

Ionizing radiation includes free protons, electrons or other charged particles above a kinetic energy of approximately 5 eV. Accordingly, alpha radiation (positively charged helium nuclei) and beta radiation (negatively charged electrons or positively charged positrons) are always ionizing radiation and are not suitable for the inventive sensor.

Since they are electrically neutral, free neutrons do not themselves have a notable interaction with electrons. However, they ionize indirectly by way of atomic reactions or scattering processes on atomic nuclei. The most effective transfer of momentum of fast neutrons occurs on hydrogen atomic nuclei which have almost the same mass (elastic impact). For this reason, fast neutrons are particularly dangerous for living tissue, because the latter always contains water (for instance in the cytosol), and are not suitable for the sensor employed in the inventive manner.

Preferably, the wavelength of the electromagnetic waves preferably used by the sensor for sampling lies in the visible range. Similarly preferably, the wavelength lies above 400 nm but below 1 mm, particularly preferably below 800 nm. Therefore, in particular, no X-ray radiation and also none of the health-endangering forms of radiation already mentioned above is used for sampling using sensors.

As already mentioned, the sensor property of the sensor is preferably based on the detection of non-ionizing electromagnetic waves, but it can also comprise other types of waves, in particular ultrasound waves, for object detection and object mapping. Any types of sensors are suitable and encompassed by the term “sensor” which can detect an outer shape of an object (that is to say of the breast). However, the technique of the sensors must not be health-endangering, in particular the detection method must not be ionizing for the tissue of the female patient.

Imaging parameters comprise adjustable parameters of the mammography system, which influence the image quality and preferably have to be selected for an optimum representation of the glandular tissue of the breast. The adjustable parameters preferably also have the indication of a specific X-ray filter employed for the current imaging. X-ray filters can be replaced depending on the requirement. “Sampling” should be taken to mean sampling with sensors, i.e. not mechanical sampling.

Particularly preferably, the sampling of the preliminary image data comprises an optical image recording of the image region or image recording region of the subsequent mammography. Preliminary image data should be taken to mean image data which does not comprise the actual mammography and is not used directly for a subsequent diagnosis, instead it is captured before the actual mammography.

Furthermore, the obtained preliminary image data is evaluated with regard to a subsequent X-ray mapping of the breast, wherein adapted values for imaging parameters are obtained for the subsequent X-ray mapping.

Finally, the mammography system is adapted on the basis of the obtained adapted values of the imaging parameters. The adaption comprises the adjustment of the imaging parameters to the obtained adapted values.

In the inventive manner, the idea of carrying out relevant calculations for preparation of the actual imaging, i.e. the mammography, at least partially as early as before the preliminary image recording, also referred to as an “X-ray advance shot”, is carried out, which comprises imaging with reduced resolution and with X-ray radiation, is pursued in order to thus reduce the time during which the female patient has to remain with their breast compressed and thereby to increase the patient comfort, or even to dispense with the preliminary image recording with X-ray radiation entirely, whereby the exposure of the patient to radiation is also reduced.

In the inventive method for carrying out a mammography of a mammography system, the inventive method for adapting a mammography of a mammography system is carried out before the actual mammography, wherein adapted values are ascertained for imaging parameters.

A mammography is subsequently carried out on the basis of the adapted values for imaging parameters ascertained and set in the inventive method for adapting a mammography of a mammography system. The inventive method for carrying out a mammography of a mammography system can be carried out with increased patient comfort compared to a conventional process because the phase in which the female patient has to wait with compressed breast for the actual imaging is reduced compared to the conventional process.

The inventive adaptation facility has a sensor unit for sampling an image region in which a breast to be mapped is positioned. Sampling occurs during the positioning and compression of the breast by way of a sensor whose function is based on a physical active principle without an ionizing effect on patient tissue.

Preferably, the sensor employs electromagnetic waves for sampling whose wavelength lies significantly outside of the wavelength range of ionizing electromagnetic waves and, in particular, above the wavelength range of X-ray radiation. Therefore, the sensor, in particular, does not use any X-ray radiation. Preliminary image data of the image region is obtained during the sampling process of the sensor.

An evaluation unit for the evaluation of the obtained preliminary image data with regard to the dynamic behavior of the breast during the image recording of the preliminary image data is also part of the inventive adaptation facility, with evaluation data being obtained. The evaluation data comprises adapted values for imaging parameters with regard to subsequent X-ray mapping of the breast.

The inventive adaptation facility also has an adaptation unit for adapting the mammography system on the basis of the obtained adapted values of the imaging parameters. The inventive adaptation facility shares the advantages of the method for adapting a mammography of a mammography system.

The inventive mammography system has a scanning unit for acquiring mammography image data from the breast of a female patient. A control facility (also referred to as a controller) for actuating the scanning unit is also part of the inventive mammography system. The inventive mammography system additionally comprises an evaluation facility for ascertaining image data on the basis of raw data of the scanning unit. In addition, the inventive mammography system comprises a control facility for actuating the scanning unit for mammography imaging and an inventive adaptation facility. The inventive mammography system shares the advantages of the inventive adaptation facility. In particular, the patient comfort of the inventive mammography system is increased owing to the reduction of the time during which the female patient has to remain with their compressed breast compared to conventional mammography systems.

A majority of said components of the inventive adaptation facility can be wholly or partially implemented in the form of software modules in a processor of a corresponding computing system, for example of a control facility of a mammography system or a computer, which is used to control such a system. An implementation largely in terms of software has the advantage that even previously used computing systems can be easily retrofitted by way of a software update in order to work in the inventive manner.

In this regard, the object is also achieved by a corresponding computer program product with a computer program, which can be loaded directly into a computing system, with program segments in order to execute the steps of the inventive method for adapting a mammography of a mammography system, in particular the steps for evaluation of the obtained preliminary image data and for adapting the mammography system when the program in the computing system. Apart from the computer program, such a computer program product can possibly comprise additional elements, such as documentation and/or additional components, also hardware components, such as hardware keys (dongles, etc.) in order to use the software.

A computer-readable medium, for example a memory stick, a hard disk or another kind of transportable or permanently installed data carrier, on which the program segments of the computer program, which can be read and executed by a computing system, are stored, can serve for transportation to the computing system and/or for storage on or in the computing system. The computing system can have, for example, one or more cooperating microprocessor(s) or the like for this purpose.

The dependent claims as well as the subsequent description respectively contain particularly advantageous embodiments and developments of the invention. In particular, the claims of one category of claim can also be developed analogously to the dependent claims of a different category of claim. In addition, within the scope of the invention, the various features of different exemplary embodiments and claims can also be combined to form new exemplary embodiments.

In a preferred variant of the inventive method for adapting a mammography of a mammography system, the sensor is based on at least one of the following sensor technologies:
Time-of-Flight sensor system,
LiDAR (light detection and ranging) sensor system.

A Time-of-Flight sensor system comprises a distance camera system for measuring distances between camera and object for each image point on the basis of the time-of-flight, the cycle time of an artificial light signal, for example of a laser or an LED. Advantageously, 3D data of the body of the patient can be obtained and the imaging parameters, in particular the collimation of the X-ray radiation and the activation of the X-ray pixels of the X-ray detector, but also the spectrum of the X-ray radiation and its intensity, can be adjusted to the ascertained 3D data.

Laser-based time-of-flight cameras belong to a broader category of scannerless LiDAR systems, in which the entire scene is captured with each laser pulse, in contrast to the point-by-point capture with a laser beam, as is customary with scanning LiDAR systems.

Preferably, during the step of sampling the image region, evaluation data about the dynamic behavior of the breast during the image recording of the preliminary image data is obtained and the step of evaluating the obtained preliminary image data comprises ascertaining values for imaging parameters, adapted to the preliminary image data, for the mammography of the breast on the basis of the evaluation data.

The evaluation of the obtained preliminary image data preferably comprises the continuous monitoring of a compression process of the breast. Observation of the compression process of the breast allows conclusions to be drawn about the proportion of the glandular tissue and its spatial distribution in the breast since the proportion of the glandular tissue correlates with the elasticity of the breast tissue. Values for the respective proportion of the glandular tissue and therewith also values for the exposure parameters may be obtained on the basis of the ascertained elasticity values. In this way, the preliminary recording with X-ray radiation, also referred to as an advance shot, can be completely left out, whereby the exposure of the female patient to radiation is reduced. Preferably, in this variant, as a preliminary recording the “advance shot” burdened by X-ray radiation is therefore completely omitted, whereby the exposure of the female patient to radiation is reduced and a further time saving is achieved, and this relates to the maintaining of the position of the female patient with compressed breast.

Particularly preferably, the evaluation comprises a segmentation of the preliminary image data. The segmentation is preferably carried out with an algorithm, which is based on artificial intelligence. However, the algorithm can also be based on specified algorithm steps which are determined by a fixed model. The segmentation separates different structures of the breast, or also outside of the breast in the surroundings of the breast, from one another and localizes them, so regions of the breast can be ascertained which are to be captured by the mammography. Other regions which are to be excluded from the mammography are similarly localized in this way. If these regions are projected onto the X-ray detector, then portions of the X-ray detector or the X-ray detector pixels present there, which correlate with regions of the breast to be mapped, can be activated and portions of the X-ray detector, which do not correlate with regions of the human body to be mapped, can be deactivated. The imaging is rendered more efficient in this way and the image calculation is accelerated as well as the exposure to X-rays reduced to a necessary amount.

Therefore, the segmentation preferably comprises at least one of the following portions of the breast and/or the surroundings of the breast:
the chest wall in relation to the breast or the pectoral muscle,
the axilla,
the breast tissue.

The chest wall of the breast or the pectoral muscle respectively and the axilla should not be mapped during the course of a mammography. By contrast, the breast tissue should be mapped. The regions of the X-ray detector corresponding therewith respectively should be deactivated or activated for the mammography accordingly.

In a preferred variant of the inventive method for adapting imaging parameters of a mammography system, the breast tissue is segmented and is projected onto the detector surface of an X-ray detector of the mammography system. The breast tissue is projected on the basis of the known extrinsic and intrinsic parameters of the sensor and of the X-ray detector. On the basis thereof, the X-ray detector pixels are calculated which map the breast tissue of interest. This technique demarcates regions on the X-ray detector which are to be activated for the mammography, and which are to be deactivated for the mammography. In the process, pixels are automatically identified which comprise breast tissue or represent the breast tissue. Pixels which, by contrast, represent tissue of other components of the body can be excluded. The accuracy of this exclusion does not have to lie in the range of the pixel size, rather it can be cruder and should rather exclude more pixels than too few in order to achieve a safety margin.

Advantageously, the otherwise usual first of the two current region-growing algorithms used for ascertaining the position and extent of the breast can be omitted since the shape of the breast tissue is already known due to the sensors. In addition, it is possible to determine that not all detector elements are read out, rather only the relevant ones, that is to say those which are located under breast tissue. As explained later, collimators can also be actuated in order to not burden regions of the body not required for the imaging with X-ray radiation.

Preferably, the evaluation of the preliminary image data comprises ascertaining and localizing body tissue which is located outside of the breast region but is located in the beam path of the X-ray beam of the mammography system. Body tissue of this kind disrupts the imaging and would be unnecessarily burdened with X-rays. Therefore, the collimation of the X-ray beam and/or the activated X-ray detector pixels is/are advantageously selected accordingly, so in the best case this body tissue is neither irradiated nor mapped.

It sometimes occurs that the ascertained and localized body tissue comprises one of the following tissue types:
the nose,
fatty tissue of the stomach,
the arm of the female patient.

Advantageously, the beam path can accordingly be adjusted by suitable adaption of the collimation of the X-ray beam such that it excludes said regions of the body of the female patient during the actual mammography, so the exposure of the female patient to radiation is reduced and the image quality of the mammography is improved. Here too, liberal exclusion of the undesired regions of the body can reliably prevent mapping of these regions.

Particularly preferably, in the inventive method for adapting a mammography of an mammography system, the step of adapting the mammography system comprises at least one of the following processes, preferably a plurality of the following processes, most particularly preferably all of the following processes:
defining the collimation parameters of the X-ray source of the mammography system for the definition of the region of the beam path of the X-ray beams,
defining the detector pixels of the X-ray detector of the mammography system, which map breast tissue,
adapting the exposure parameters of the X-ray source of the mammography system, wherein the exposure parameters comprise at least one of the following types of parameter:
the hardness of the X-ray spectrum, and therewith the electric voltage Ua between anode and cathode of the X-ray source of the mammography system,
the heating voltage Uh of the cathode.

Advantageously, the imaging is already set on the basis of the preliminary image data during the compression of the breast of the female patient, so the setup time of the mammography is reduced and the comfort for the female patient, who does not have to stay for as long as usual with breast compressed, is improved.

In a preferred variant of the inventive method for carrying out a mammography, the exposure parameters are obtained by way of a preliminary X-ray recording and the remaining imaging parameters are obtained by way of the inventive method for adapting a mammography of a mammography system. In this variant, some of the calculations of the imaging parameters are therefore ascertained on the basis of the “advance shot” and some of the results of the calculations, which deal with the geometry of the imaging, are ascertained on the basis of the X-ray beam-free preliminary recording.

As already mentioned, the imaging parameters can alternatively also be adapted solely on the basis of the X-ray beam-free preliminary recording. This may be the case if the sensor employed for this or the sensors employed for this observe the entire compression process and conclusions can thus be drawn about the proportion of the glandular tissue in the breast on the basis of elasticity measurements. The values typical for this breast thickness and breast elasticity can be ascertained on the basis of real comparison recordings.

Lowering the compression paddle onto the breast increases (for example linearly) the mechanical force which acts on the breast by way of the compression paddle. It is possible to estimate the breast elasticity on the basis of the deformation of the breast as the force increases. This estimate can be made, for example, via a look-up table, which contains a change in force, based on the breast area, and elastic properties. The values of the look-up table can be obtained, for example, via a finite element simulation.

The observed deformation, which goes with the breast area/volume, can then be used to estimate the proportion of glandular tissue in the breast. The proportion of the glandular tissue can then be used to predict the X-ray absorption via a further look-up table or simulation and to therewith select the exposure parameters or imaging parameters accordingly.

In the inventive method for carrying out a mammography, data in respect of a underexposure or overexposure or, generally, data in respect of a deviation of the adapted values of the imaging parameters from ideal values can be collected even after the mammography which has occurred and this data can be reported back to the algorithm used for the evaluation and adaptation, so the result of the adaptation in the next mammography is improved further. A preliminary recording with X-rays which has been carried out can also be used to check the adaptation on the basis of the X-ray beam-free preliminary recording in order to further improve the result of the mammography.

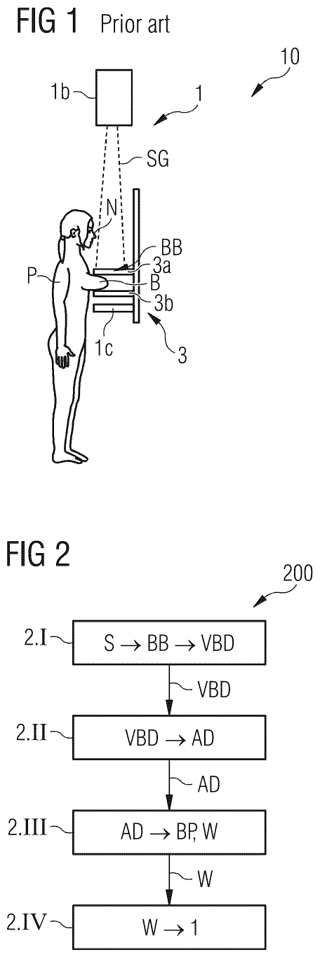

FIG. 1 shows a schematic representation 10 of a scenario of a conventional recording of mammography image data BD of the breast B of a female patient P. The female patient P is standing in front of a mammography system 1, with their breast B being inserted into a compression facility 3 and being compressed between the compression plates 3a, 3b thereof (an upper compression plate 3a and a lower compression plate 3b). The mammography system 1 has an X-ray source 1b represented at the top of the image and an X-ray detector 1c represented at the bottoms of the image. The X-ray source 1b emits X-ray radiation in a beam path SG in the direction of the breast B or in the direction of an image region BB comprising the breast B. Some of the X-ray radiation penetrates the breast B and is captured by the X-ray detector 1c. As can be seen in FIG. 1, the nose N of the female patient P is located in the beam path SG of the emitted X-ray radiation. As is explained later, it is desirable that the nose N of the female patient P is not mapped on a mammography image generated by the X-ray detector 1c. Conventionally, a preliminary mapping referred to as an “advance shot” is carried out with X-ray radiation before the actual mammography. If the nose N of the female patient P can be seen on the preliminary mapping, the collimation of the X-ray radiation emitted by the X-ray source 1b is adjusted accordingly, so the beam path SG excludes the nose N of the female patient P. Settings relating to the hardness of the X-ray spectrum (electric voltage Ua) and the intensity of the X-ray spectrum (heating voltage Uh) are conventionally carried out solely on the basis of this preliminary mapping. As already explained, after the preliminary mapping, the female patient P has to wait with compressed breast until the values have been calculated for said settings. Only then can the actual mammography be started. The inventive method illustrated in FIG. 2 should accordingly reduce the waiting period and therewith also reduce the period during which the female patient P has to stay in this uncomfortable situation with compressed breast B.

FIG. 2 shows a flowchart 200 which illustrates a method for adapting a mammography of a mammography system 1 according to a first exemplary embodiment of the invention.

In the method for adapting a mammography of an mammography system 1, in step 2.I, an image region BB is sampled in which a breast B to be mapped is positioned. This sampling occurs during the positioning and compression of the breast B. The sampling is carried out by an optical sensor S, with preliminary image data VBD being obtained of the image region BB. As already explained, this optical mapping can occur in addition to an “advance shot” with X-ray radiation or replace this “advance shot”.

In step 2.II, the obtained preliminary image data VBD is evaluated with regard to the dynamic behavior of the breast B (see FIG. 1) during the image recording of the preliminary image data VBD, with evaluation data AD being obtained. The dynamic behavior of the breast B produces items of information about the elasticity of the breast B, which in turn correlates with the distribution and density of the glandular tissue of the breast B. The glandular tissue of the breast B should be mapped with optimum quality during the subsequent mammography.

In step 2.III, values W for imaging parameters BP, adapted to the optically captured preliminary image data VBD, for the mammography of the breast B are ascertained on the basis of the evaluation data AD.

In step 2.IV, finally, the mammography system 1 is adapted by way of the setting with the adapted values W for the imaging parameters BP. In particular, the collimation for the definition of the region of the beam path of the X-ray radiation in the mammography is defined. This measure prevents the mapping of body parts on the mammography image which do not form part of the breast. Furthermore, detector pixels are also selected which are required for the mapping of the glandular tissue of the breast. Detector pixels which, owing to the adapted collimation of the X-ray radiation field SG, are not required for the mammography imaging, are also not activated. In this way, the image reconstruction is limited to the required number of detector pixels. Furthermore, exposure parameters, in particular the hardness of the X-ray spectrum and the heating voltage, are adapted to the ascertained density and distribution of the glandular tissue. This adaption process optimizes the quality of the subsequent mammography. As already mentioned, some of this adaption can also occur on the basis of an additional preliminary imaging via X-rays.

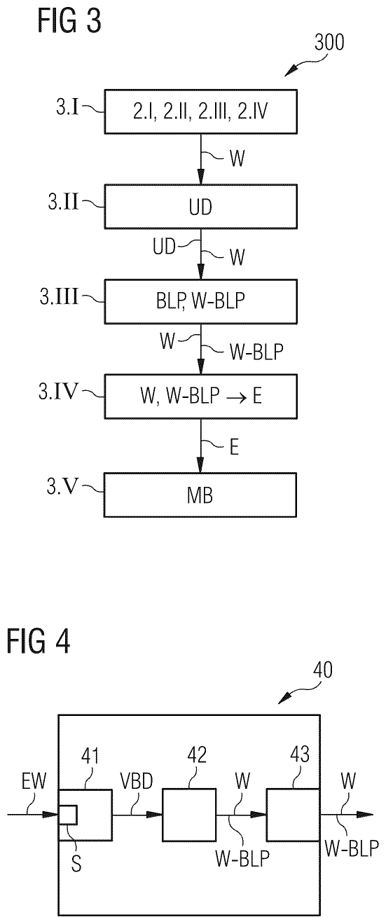

FIG. 3 shows a flowchart 300, which illustrates a method for carrying out a mammography according to an exemplary embodiment of the invention.

In step 3.I, the inventive method for adapting a mammography of a mammography system 1 is carried out with steps 2.I to 2.IV.

Furthermore, a preliminary X-ray recording occurs in step 3.II, with overview data UD being obtained in the form of low-resolution X-ray image data. In the exemplary embodiment illustrated in FIG. 3, a pre-recording with X-rays therefore occurs in addition to the optical recording of preliminary image data VBD. Such a preliminary X-ray recording occurs with a low radiation dose since image quality is not crucial.

In step 3.III, values W-BLP are obtained for exposure parameters BLP of a subsequent mammography imaging of the breast B of the female patient P. The values W-BLP are ascertained on the basis of the items of information UD, W obtained by the in the preliminary recordings. In this embodiment, the design of the X-ray beam field or of the beam path SG and the selection of the X-ray pixels for the subsequent mammography occurs on the basis of the adaptation carried out in step 3.I. By contrast, the exposure is set on the basis of the different preliminary recordings which occurred in the two steps 3.I and 3.II.

In step 3.IV, setting E of the exposure parameters BLP occurs on the basis of the values W-BP of imaging parameters ascertained in step 3.III and on the basis of the remaining imaging parameters, which were obtained on the basis of the items of information ascertained in step 3.I or the items of information ascertained in the inventive method for adapting a mammography of a mammography system.

In step 3.V, mammography imaging MB occurs on the basis of the setting E of the imaging parameters BP which occurred in step 3.IV and, in particular, the ascertained exposure parameters BLP.

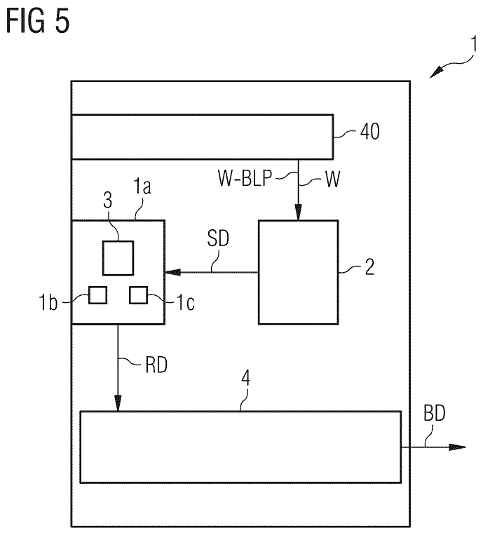

FIG. 4 shows a schematic representation of an adaptation facility 40 according to an exemplary embodiment of the invention.

The adaptation facility 40 has a sensor unit 41 with an optical sensor S for sampling an image region BB (see FIG. 1). A breast B to be mapped is positioned in the image region BB. Sampling by way of the sensor S of the sensor unit 41 occurs during the positioning and compression of the breast B. The sensor S does not use X-ray radiation, rather electromagnetic waves EW in the optically visible range, for sampling. Preliminary image data VBD of the image region BB is obtained by way of the sampling process.

An evaluation unit 42 for evaluation of the obtained preliminary image data VBD with regard to the dynamic behavior of the breast B during the image recording of the preliminary image data VBD is also part of the adaptation facility 40. Adapted values W for imaging parameters BP and, in particular, also values W-BLP for the exposure parameters for subsequent X-ray imaging are ascertained during the evaluation.

The ascertained adapted values W, W-BLP are transmitted to an adaptation unit 43, which is similarly part of the adaptation facility 40 and is configured to set a mammography system 1 (see FIG. 5) on the basis of the adapted values W, W-BLP, in particular as regards its exposure parameters BLP.

FIG. 5 illustrates a schematic representation of a mammography system 1 according to an exemplary embodiment of the invention.

The mammography system 1 has a scanning unit 1a with a compression facility 3, an X-ray source 1b and an X-ray detector 1c.

A control facility 2 for actuating the scanning unit 1a with the aid of control data SD for the acquisition of mammography image data BD is also part of the mammography system 1. The mammography system 1 also comprises the adaptation facility 40 illustrated in FIG. 4 for the adaption of imaging parameters of the scanning unit 1a on the basis of adapted values W of imaging parameters and, in particular, values W-BLP of exposure parameters BLP. The mammography system 1 comprises, moreover, an image-ascertaining facility (also referred to as an image processor) 4 for ascertaining mammography image data BD on the basis of raw data RD of the scanning unit 1a.

To conclude, reference is made once again to the fact that the above-described method and apparatuses are merely preferred exemplary embodiments of the invention and that the invention can be varied by a person skilled in the art without departing from the scope of the invention insofar as it is specified by the claims. For the sake of completeness, reference is also made to the fact that use of the indefinite article “a” or “an” does not preclude the relevant features from also being present multiple times. Similarly, the term “unit” does not preclude this from being composed of a plurality of components, which can possibly also be spatially distributed. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as an data processing system)or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for adapting a mammography of a mammography system, the method comprising:
sampling an image region using a sensor during a positioning and a compression of a breast, the sensor operating based on a physical active principle without an ionizing effect on patient tissue, and obtaining preliminary image data from the image region;
evaluating the obtained preliminary image data with regard to a subsequent X-ray mapping of the breast, and obtaining adapted values for imaging parameters for the subsequent X-ray mapping; and
adapting the mammography system based on the obtained adapted values of the imaging parameters.

2. The method of claim 1, wherein the subsequent X-ray mapping comprises at least one of the following X-ray mappings:
a preliminary X-ray recording of the breast, or
a mammography recording of the breast.

3. The method of claim 1, wherein during the sampling the image region, evaluation data about a dynamic behavior of the breast during an image recording of the preliminary image data is obtained and the evaluating the obtained preliminary image data comprises ascertaining values for imaging parameters for the mammography of the breast based on the evaluation data, the ascertained values adapted to the preliminary image data.

4. The method of claim 1, wherein the sensor is based on one of the following sensor technologies:
a Time-of-Flight sensor system, or
a Lidar sensor system.

5. The method of claim 1, wherein the evaluating comprises a segmentation of the preliminary image data.

6. The method of claim 5, wherein the segmentation comprises at least one of the following:
a pectoral muscle,
an axilla, or
breast tissue.

7. The method of claim 6, wherein the breast tissue is segmented and projected onto a detector surface of an X-ray detector of the mammography system.

8. The method of claim 1, wherein the evaluating comprises ascertaining and localizing tissue outside of the breast region and in a beam path of an X-ray beam of the mammography system.

9. The method of claim 3, wherein the ascertaining values comprises defining at least one of the following types of parameter values:
collimation parameters for a definition of a region of abeam path of X-rays,
detector pixels which map breast tissue, or
exposure parameters, the exposure parameters being incorporated by the imaging parameters.

10. A method for performing a mammography, the method comprising:
performing the method of claim 1; and
performing the mammography based on the adapted values.

11. The method of claim 10, wherein exposure parameters are obtained using a preliminary X-ray recording.

12. An adaptation system, comprising:
a sensor configured to sample an image region during a positioning and a compression of a breast, the sensor configured to operate based on a physical active principle without an ionizing effect on patient tissue, wherein preliminary image data is obtained from the image region;
an evaluation unit configured to evaluate the obtained preliminary image data with regard to a subsequent X-ray mapping of the breast, wherein adapted values for imaging parameters are obtained for the subsequent X-ray mapping; and
an adaptation unit configured to adapt a mammography system based on the obtained adapted values of the imaging parameters.

13. A mammography system, comprising:
a scanning unit configured to acquire mammography image data from a breast of a female patient;
a controller configured to actuate the scanning unit;
an image processor configured to ascertain image data based on raw data of the scanning unit; and
the adaption system of claim 12.

14. A non-transitory computer program product, comprising instructions which, when executed by a computer, cause the computer to perform the method of claim 1.

15. A non-transitory computer-readable storage medium, comprising instructions which, when executed by a computer, cause the computer to perform the method of claim 1.

16. The method of claim 9, wherein the ascertaining values comprises defining all of the types of parameter values.

17. The method of claim 9, wherein the exposure parameters comprise:
a hardness of an X-ray spectrum, and
a heating voltage of a cathode.

18. The method of claim 2, wherein during the sampling the image region, evaluation data about a dynamic behavior of the breast during an image recording of the preliminary image data is obtained and the evaluating the obtained preliminary image data comprises ascertaining values for imaging parameters for the mammography of the breast based on the evaluation data, the ascertained values adapted to the preliminary image data.

19. The method of claim 2, wherein the evaluating comprises a segmentation of the preliminary image data.

20. The method of claim 2, wherein the evaluating comprises ascertaining and localizing tissue outside of the breast region and in a beam path of an X-ray beam of the mammography system.
